(19) United States
(12) Patent Application Publication
(10) Pub. No.: US 2010/0112615 A1
(43) Pub. Date: May 6, 2010

(54) GLYCOSYLTRANSFERASE ACTIVITY

(75) Inventors: Eng Kiat Lim, York (GB); Dianna Bowles, York (GB)

Correspondence Address: SPECKMAN LAW GROUP PLLC, 1201 THIRD AVENUE, SUITE 330, SEATTLE, WA, 98101, US

(73) Assignee: THE UNIVERSITY OF YORK, York (GB)

(21) Appl. No.: 11/917,326

(22) PCT Filed: Jun. 8, 2006
(86) PCT No.: PCT/GB06/02091

§ 371 Date: Jan. 7, 2010

(30) Foreign Application Priority Data
Jun. 16, 2005 (GB) 0512224.7
Oct. 27, 2005 (GB) 0521886.2

(51) Int. Cl. C12Q 1/48; C12N 1/21; C12P 19/26; C12P 17/06; C12M 1/00

(52) U.S. Cl. 435/15; 435/252.3; 435/84; 435/125; 435/289.1

(57) ABSTRACT

We describe the production of nucleotide sugars other than uridine diphosphate glucose (UDP-glucose), for example UDP-rhamnose, and the use of these nucleotide sugars in the modification of acceptor molecules.

The invention relates to the production of nucleotide sugars other than uridine diphosphate glucose (UDP-glucose), for example UDP-rhamnose, and the use of these nucleotide sugars in the modification of acceptor molecules.

Glycosyltransferases (GTases) are enzymes that post-translationally transfer glycosyl residues from an activated nucleotide sugar to monomeric and polymeric acceptor molecules such as other sugars, proteins, lipids and other organic substrates. These glycosylated molecules take part in diverse metabolic pathways and processes. The transfer of a glycosyl moiety can alter the acceptor's bioactivity, solubility and transport properties within the cell and throughout the plant. The most common activated nucleotide sugar is UDP-glucose which is used by a large number of glucosyltransferase enzymes. Examples of other GTases include rhamnosyltransferases, fucosyltransferases, sialyltransferases and galactosyltransferases each of which use a different donating nucleotide sugar. A large family of GTases in higher plants are described in our earlier application WO01/59140, which is incorporated by reference (also see Lim et al Journal Biological Chemistry 277(1): 586-92 (2002); Ross et al Genome Biology 2001 2(2): 3004.1-6, each of which are incorporated by reference) and are characterised by the presence of a C-terminal consensus sequence. The GTases of this family function in the cytosol of plant cells and catalyse the transfer of glucose to small molecular weight substrates, such as for example, phenylpropanoid derivatives, coumarins, flavanoids, other secondary metabolites and molecules known to act as plant hormones.

In addition to the glucosyltransferases disclosed in WO01/59140 other glycosyltransferases are known. For example, rhamnosyltransferases are disclosed in WO94/03591 which are flavanoid modifying enzymes that are involved in the production of pigment molecules in plants, specifically a UDP-rhamnose: anthocyanidin-3-O-rhamnoside rhamnosyltransferase. A further rhamnosyltransferase is disclosed in US2005089882 which is shown to have flavone-7-O-glucoside-2-O-rhamnosyltransferase catalytic activity and its use in the conversion of hesperdin found in orange peel to the sweetener neohesperidin. Bacterial rhamnosylransferases have also been described in transgenic plants and their use in phytoremediation of heavy metals and hydrocarbons, see WO2004050882.

In our co-pending application (WO2004/106508) we describe a whole cell biocatalyst that modifies compounds in a stereospecific fashion. Moreover, the in vitro cell based bioreactor utilises glycosyltransferases to add glucosyl moieties to compounds such as cytokinins and quercetin. We find that the bioreactor does not require an exogenous supply of UDP-glucose, (a substrate for these enzymes) this being provided by the cell that is transfected with the GTase nucleic acid molecules.

The present application relates to plant rhamnose synthase (RHM) that use UDP-glucose as substrate to form UDP-rhamnose. We have amplified three Arabidopsis RHM genes (RHM1, RHM2 and RHM3) from a cDNA library, and expressed them in E. coli cells. LC-MS analysis indicates that there is a significant increase in TDP-rhamnose level in the bacterial cells expressing RHM cDNAs compared to those without RHM cDNAs. In addition, UDP-rhamnose, which is not found in E. coli, is also accumulated in the same level as TDP-rhanmose in the cells expressing RHM genes. When Arabidopsis GT78D1, which is an example of a plant rhamnosyltransferase, was co-expressed with RHM1 cDNA in E. coli, the bacterial cells were found to synthesize quercetin-rhamnoside using the quercetin substrate added to the culture medium. This forms the basis of a means to produce novel nucleotide sugars and the modification of acceptor molecules by said nucleotide sugars to form novel acceptor: sugar combinations.

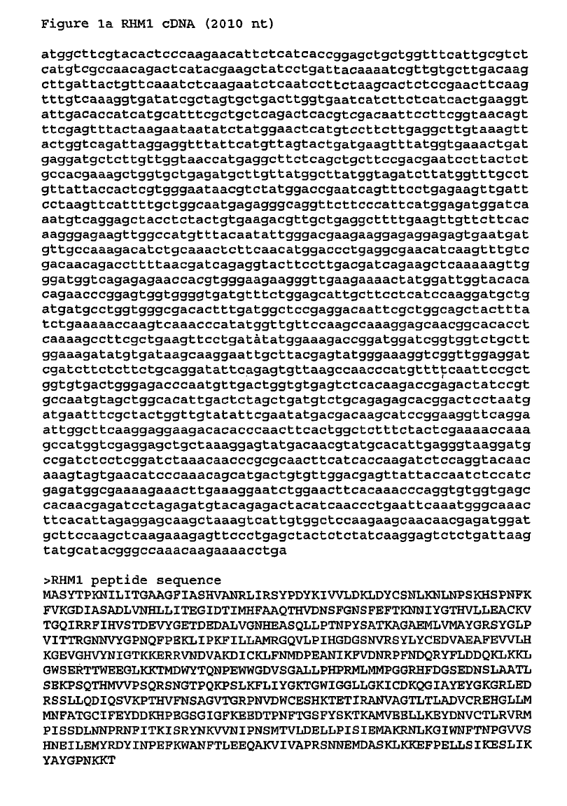

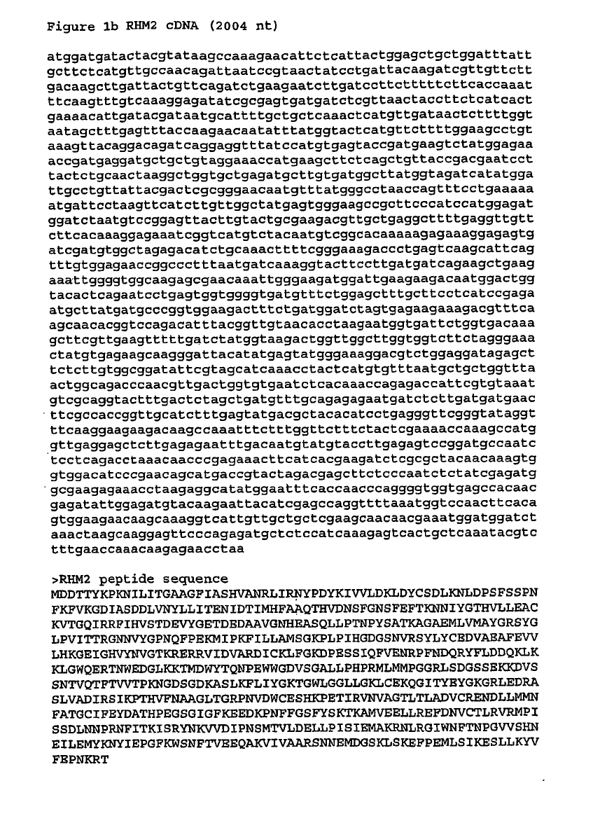

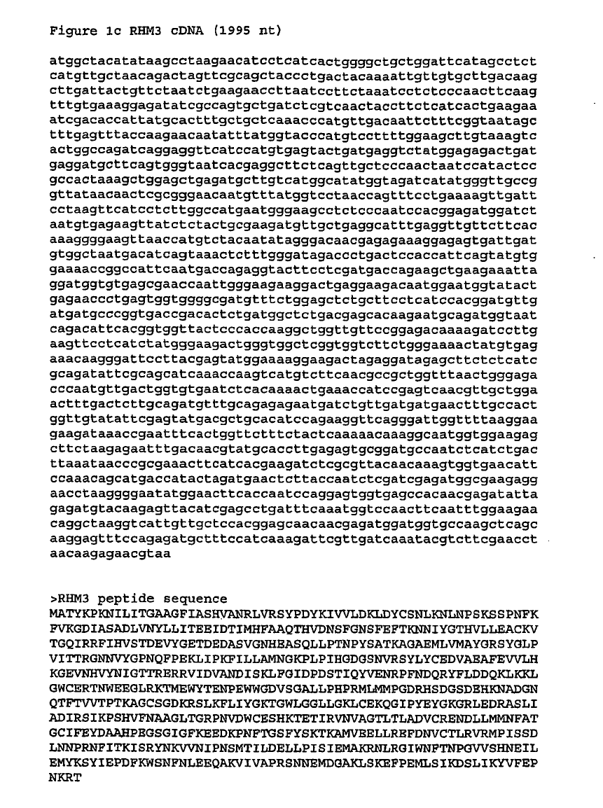

According to a first aspect of the invention there is provided a cell that is transfected with at least one nucleic acid molecule that comprises a nucleic acid sequence selected from the group consisting of:
i) a nucleic acid molecule consisting of a nucleic acid sequence as represented in FIGS. 1a, 1b or 1c;
ii) a nucleic acid molecule consisting of a nucleic acid sequence that hybridises under stringent hybridisation conditions to the nucleic acid molecules in (i) and which have rhamnose synthase activity.

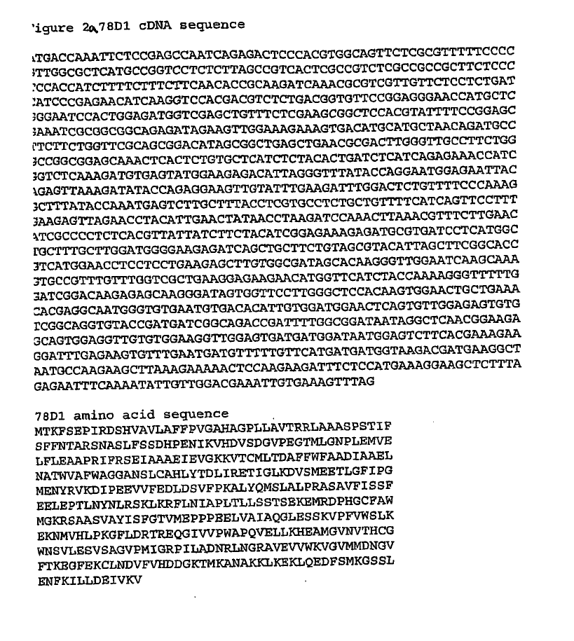

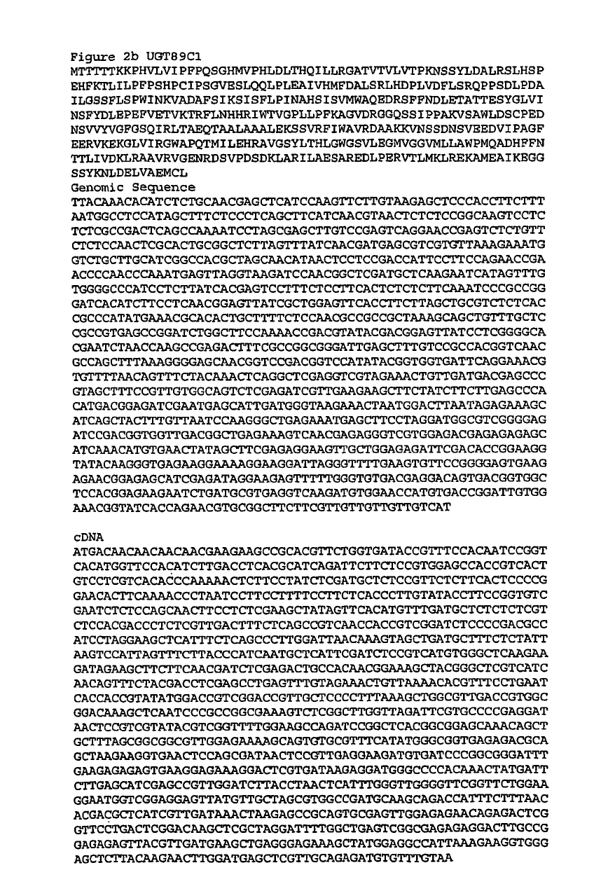

In a preferred embodiment of the invention said cell is further transfected with a nucleic acid molecule consisting of a nucleic acid sequence as represented by the nucleic acid sequences in FIG. 2a or 2b; or a nucleic acid molecule consisting of a nucleic acid sequences that hybridises under stringent hybridisation conditions to the nucleic acid molecules in FIG. 2a or 2b and which have glucosyltransferase activity.

In a further preferred embodiment of the invention said nucleic acid molecules are adapted for expression of both said rhamnose synthase and glucosyltransferase polypeptides.

Hybridization of a nucleic acid molecule occurs when two complementary nucleic acid molecules undergo an amount of hydrogen bonding to each other. The stringency of hybridization can vary according to the environmental conditions surrounding the nucleic acids, the nature of the hybridization method, and the composition and length of the nucleic acid molecules used. Calculations regarding hybridization conditions required for attaining particular degrees of stringency are discussed in Sambrook et al., Molecular Cloning: A Laboratory Manual (Cold Spring Harbor Laboratory Press, Cold Spring Harbor, N.Y., 2001); and Tijssen, Laboratory Techniques in Biochemistry and Molecular Biology—Hybridization with Nucleic Acid Probes Part I, Chapter 2 (Elsevier, N.Y., 1993). The Tₘ is the temperature at which 50% of a given strand of a nucleic acid molecule is hybridized to its complementary strand. The following is an exemplary set of hybridization conditions and is not limiting:

Very High Stringency (Allows Sequences that Share at Least 90% Identity to Hybridize)

Hybridization: 5×SSC at 65° C. for 16 hours

Wash twice: 2×SSC at room temperature (RT) for 15 minutes each

Wash twice: 0.5×SSC at 65° C. for 20 minutes each

High Stringency (Allows Sequences that Share at Least 80% Identity to Hybridize)

Hybridization: 5×−6×SSC at 65° C.-70° C. for 16-20 hours

Wash twice: 2×SSC at RT for 5-20 minutes each

Wash twice: 1×SSC at 55° C.-70° C. for 30 minutes each

Low Stringency (Allows Sequences that Share at Least 50% Identity to Hybridize)

Hybridization: 6×SSC at RT to 55° C. for 16-20 hours

Wash at least twice: 2×−3×SSC at RT to 55° C. for 20-30 minutes each.

In a preferred embodiment of the invention said nucleic acid molecule comprises a nucleic acid sequence that has at least or greater than 10% homology to the nucleic acid sequence represented in FIGS. 1a, 1b or 1c. Preferably said homology is at least 20%, 25%, 30%, 35%, 40%; 45%, 50%; 55%, 60%; 65%, 70%; 75%, 80%; 85%; 90%; 95% or at least 99% identity with the nucleic acid sequence represented in FIGS. 1a, 1b or 1c or the amino acid sequence disclosed in FIGS. 1a, 1b or 1c.

In a preferred embodiment of the invention said nucleic acid molecule comprises a nucleic acid sequence that has at least or greater than 10% homology to the nucleic acid sequence represented in FIG. 2a or 2b. Preferably said homology is at least 20%, 25%, 30%, 35%, 40%; 45%, 50%; 55%, 60%; 65%, 70%; 75%, 80%; 85%; 90%; 95% or at least 99% identity with the nucleic acid sequence represented in FIG. 2a or 2b or the amino acid sequence represented in FIG. 2a or 2b.

In a preferred embodiment of the invention said cell is a prokaryotic cell, preferably a bacterial cell.

In a further preferred embodiment of the invention said bacterial cell is a Gram negative bacterial cell, for example Escherichia coli.

In an alternative preferred embodiment of the invention said bacterial cell is a Gram positive bacterial cell, for example, a bacterium of the genus Bacillus spp. (e.g. B. subtilis; B. licheniformis; B. amyloliquefaciens).

Gram positive and Gram negative bacteria differ in many respects from one another. A difference exists in the nature of their respective cell walls. The biochemical composition of the B. subtilis cell wall is quite different from that of E. coli. The cell walls of E. coli and B. subtilis contain a framework that is composed of peptidoglycan, a complex of polysaccharide chains covalently cross-linked by peptide chains. This forms a semi-rigid structure that confers physical protection to the cell since the bacteria have a high internal osmotic pressure and can be exposed to variations in external osmolarity. In Gram-positive bacteria, such as the members of the genus Bacillus, the peptidoglycan framework may represent as little as 50% of the cell wall complex and these bacteria are characterised by having a cell wall that is rich in accessory polymers such as teichoic acids. Methods to transform bacteria are well known in the art and have been established for many years. These include chemical methods (e.g. calcium permeabilization) or physical permeabilization (e.g. electroporation).

In an alternative preferred embodiment of the invention said cell is a eukaryotic cell.

Preferably said eukaryotic cell is selected from the group consisting of: a yeast cell; an insect cell; a mammalian cell or a plant cell.

In a preferred embodiment of the invention said cell is a plant cell.

In a preferred embodiment of the invention said plant cell is selected from: corn (Zea mays), canola (Brassica napus, Brassica rapa ssp.), alfalfa (Medicago sativa), rice (Oryza sativa), rye (Secale cerale), sorghum (Sorghum bicolor, Sorghum vulgare), sunflower (helianthus annuas), wheat (Tritium aestivum), soybean (Glycine max), tobacco (Nicotiana tabacum), potato (Solanum tuberosum), peanuts (Arachis hypogaea), cotton (Gossypium hirsutum), sweet potato (Iopmoea batatus), cassava (Manihot esculenta), coffee (Cofea spp.), coconut (Cocos nucifera), pineapple (Anana comosus), citris tree (Citrus spp.) cocoa (Theobroma cacao), tea (Camellia senensis), banana (Musa spp.), avacado (Persea americana), fig (Ficus casica), guava (Psidium guajava), mango (Mangifer indica), olive (Olea europaea), papaya (Carica papaya), cashew (Anacardium occidentale), macadamia (Macadamia intergrifolia), almond (Prunus amygdalus), sugar beets (Beta vulgaris), oats, barley, vegetables and ornamentals.

Preferably, plant cells of the present invention are crop plant cells (for example, cereals and pulses, maize, wheat, potatoes, tapioca, rice, sorghum, millet, cassava, barley, pea, and other root, tuber or seed crops. Important seed crops are oil-seed rape, sugar beet, maize, sunflower, soybean, and sorghum. Horticultural plants to which the present invention may be applied may include lettuce, endive, and vegetable brassicas including cabbage, broccoli, and cauliflower, and carnations and geraniums. The present invention may be applied in tobacco, cucurbits, carrot, strawberry, sunflower, tomato, pepper, chrysanthemum.

Grain plants that provide seeds of interest include oil-seed plants and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava been, lentils, chick pea.

In a preferred embodiment of the invention said nucleic acid molecule comprises the nucleic acid sequence as presented in FIGS. 1a, 1b or 1c. Preferably said nucleic acid molecule consists of the nucleic acid sequence as presented in FIGS. 1a, 1b or 1c.

In a further preferred embodiment of the invention said nucleic acid molecule comprises the nucleic acid sequence as presented in FIG. 2a or 2b. Preferably said nucleic acid molecule consists of the nucleic acid sequence as presented in FIG. 2a or 2b.

In a preferred embodiment of the invention said cell is transfected with a vector, preferably an expression vector that includes said nucleic acid molecules that encode said rhamnose synthase and said glucosyltransferase polypeptides and is adapted for the expression of same.

Typically said adaptation includes, by example and not by way of limitation, the provision of transcription control sequences (promoter sequences) that mediate cell specific expression. These promoter sequences may be cell specific, inducible or constitutive.

Promoter is an art recognised term and, for the sake of clarity, includes the following features which are provided by example only. Enhancer elements are cis acting nucleic acid sequences often found 5′ to the transcription initiation site of a gene (enhancers can also be found 3′ to a gene sequence or even located in intronic sequences and is therefore position independent). Enhancers function to increase the rate of transcription of the gene to which the enhancer is linked. Enhancer activity is responsive to trans acting transcription factors which have been shown to bind specifically to enhancer elements. The binding/activity of transcription factors (please see Eukaryotic Transcription Factors, by David S Latchman, Academic Press Ltd, San Diego) is responsive to a number of environmental cues that include, by example and not by way of limitation, intermediary metabolites (e.g. sugars), environmental effectors (e.g. light).

Promoter elements also include so called TATA box and RNA polymerase initiation selection (RIS) sequences that function to select a site of transcription initiation. These sequences also bind polypeptides that function, inter alia, to facilitate transcription initiation selection by RNA polymerase.

Adaptations also include the provision of selectable markers and autonomous replication sequences which both facilitate the maintenance of said vector in either the eukaryotic cell or prokaryotic host. Vectors that are maintained autonomously are referred to as episomal vectors. Episomal vectors are desirable since these molecules can incorporate large DNA fragments (30-50 kb DNA).

Adaptations which facilitate the expression of vector encoded genes include the provision of transcription termination/polyadenylation sequences. This also includes the provision of internal ribosome entry sites (IRES) that function to maximise expression of vector encoded genes arranged in bicistronic or multi-cistronic expression cassettes.

There is a significant amount of published literature with respect to expression vector construction and recombinant DNA techniques in general. Please see, Sambrook et al (1989) Molecular Cloning: A Laboratory Manual, Cold Spring Harbour Laboratory, Cold Spring Harbour, N.Y. and references therein; Marston, F (1987) DNA Cloning Techniques: A Practical Approach Vol III IRL Press, Oxford UK; DNA Cloning: F M Ausubel et al, Current Protocols in Molecular Biology, John Wiley & Sons, Inc.(1994).

According to a further aspect of the invention there is provided a plant comprising a plant cell according to the invention.

According to a further aspect of the invention there is provided a seed comprising a plant cell according to the invention.

According to a further aspect of the invention there is provided a cell culture comprising a cell according to the invention.

According to a further aspect of the invention there is provided the use of a cell according to the invention in the production of nucleotide sugars.

In a preferred embodiment of the invention said nucleotide sugar is UDP-rhamnose or dTDP-rhamnose.

According to a further aspect of the invention there is provided a method for the production of a nucleotide sugar comprising the steps of:
i) providing a cell culture according to the invention and rhamnose;
ii) culturing said cell under cell culture conditions that facilitate the production of a nucleotide sugar wherein said nucleotide sugar is UDP-rhamnose; and optionally
iii) separating or purifying UDP rhamnose from the cell or cell culture media.

According to an aspect of the invention there is provided a method for the production of a substrate which is modified with a rhamnoside sugar comprising the steps of:
i) providing a cell culture according to the invention and at least one substrate to be modified;
ii) culturing said cell under cell culture conditions that facilitate the production of a sugar modified substrate; and optionally
iii) separating or purifying said sugar modified substrate from the cell or cell culture media.

In a preferred embodiment of the invention said substrate is quercetin.

According to a further aspect of the invention there is provided a reaction vessel comprising a cell according to the invention.

In a preferred embodiment of the invention said vessel is a bioreactor.

In a further preferred embodiment of the invention said bioreactor comprises nutrient media that does not include an exogenous supply of UDP-glucose.

Bioreactors, for example fermentors, are vessels that comprise cells or enzymes and typically are used for the production of molecules on an industrial scale. The molecules can be recombinant proteins (e.g. enzymes such as proteases, lipases, amylases, nucleases, antibodies) or compounds that are produced by the cells contained in the vessel or via enzyme reactions that are completed in the reaction vessel. Typically, cell based bioreactors comprise the cells of interest and include all the nutrients and/or co-factors necessary to carry out the reactions.

If bacteria are used in the process according to the invention, they are grown or cultured in the manner with which the skilled worker is familiar, depending on the host organism. As a rule, bacteria are grown in a liquid medium comprising a carbon source, usually in the form of sugars, a nitrogen source, usually in the form of organic nitrogen sources such as yeast extract or salts such as ammonium sulfate, trace elements such as salts of iron, manganese and magnesium and, if appropriate, vitamins, at temperatures of between 0° C. and 100° C., preferably between 10° C. and 60° C., while gassing in oxygen.

The pH of the liquid medium can either be kept constant, that is to say regulated during the culturing period, or not. The cultures can be grown batchwise, semi-batchwise or continuously. Nutrients can be provided at the beginning of the fermentation or fed in semi-continuously or continuously. The products produced can be isolated from the bacteria as described above by processes known to the skilled worker, for example by extraction, distillation, crystallization, if appropriate precipitation with salt, and/or chromatography. To this end, the organisms can advantageously be disrupted beforehand. In this process, the pH value is advantageously kept between pH 4 and 12, preferably between pH 6 and 9, especially preferably between pH 7 and 8.

The culture medium to be used must suitably meet the requirements of the strains in question. Descriptions of culture media for various microorganisms can be found in the textbook “Manual of Methods for General Bacteriology” of the American Society for Bacteriology (Washington D.C., USA, 1981).

As described above, these media which can be employed in accordance with the invention usually comprise one or more carbon sources, nitrogen sources, inorganic salts, vitamins and/or trace elements.

Preferred carbon sources are sugars, such as mono-, di- or polysaccharides. Examples of carbon sources are glucose, fructose, mannose, galactose, ribose, sorbose, ribulose, lactose, maltose, sucrose, raffinose, starch or cellulose. Sugars can also be added to the media via complex compounds such as molasses or other by-products from sugar refining. The addition of mixtures of a variety of carbon sources may also be advantageous. Other possible carbon sources are oils and fats such as, for example, soya oil, sunflower oil, peanut oil and/or coconut fat, fatty acids such as, for example, palmitic acid, stearic acid and/or linoleic acid, alcohols and/or polyalcohols such as, for example, glycerol, methanol and/or ethanol, and/or organic acids such as, for example, acetic acid and/or lactic acid.

Nitrogen sources are usually organic or inorganic nitrogen compounds or materials comprising these compounds. Examples of nitrogen sources comprise ammonia in liquid or gaseous form or ammonium salts such as ammonium sulfate, ammonium chloride, ammonium phosphate, ammonium carbonate or ammonium nitrate, nitrates, urea, amino acids or complex nitrogen sources such as cornsteep liquor, soya meal, soya protein, yeast extract, meat extract and others. The nitrogen sources can be used individually or as a mixture.

Inorganic salt compounds which may be present in the media comprise the chloride, phosphorus and sulfate salts of calcium, magnesium, sodium, cobalt, molybdenum, potassium, manganese, zinc, copper and iron.

Inorganic sulfur-containing compounds such as, for example, sulfates, sulfites, dithionites, tetrathionates, thiosulfates, sulfides, or else organic sulfur compounds such as mercaptans and thiols may be used as sources of sulfur for the production of sulfur-containing fine chemicals, in particular of methionine.

Phosphoric acid, potassium dihydrogenphosphate or dipotassium hydrogenphosphate or the corresponding sodium-containing salts may be used as sources of phosphorus.

Chelating agents may be added to the medium in order to keep the metal ions in solution. Particularly suitable chelating agents comprise dihydroxyphenols such as catechol or protocatechuate and organic acids such as citric acid.

The fermentation media used according to the invention for culturing microorganisms usually also comprise other growth factors such as vitamins or growth promoters, which include, for example, biotin, riboflavin, thiamine, folic acid, nicotinic acid, panthothenate and pyridoxine. Growth factors and salts are frequently derived from complex media components such as yeast extract, molasses, cornsteep liquor and the like. It is moreover possible to add suitable precursors to the culture medium. The exact composition of the media compounds heavily depends on the particular experiment and is decided upon individually for each specific case. Information on the optimization of media can be found in the textbook “Applied Microbiol. Physiology, A Practical Approach” (Editors P. M. Rhodes, P. F. Stanbury, IRL Press (1997) pp. 53-73, ISBN 0 19 963577 3). Growth media can also be obtained from commercial suppliers, for example Standard 1 (Merck) or BHI (brain heart infusion, DIFCO) and the like.

All media components are sterilized, either by heat (20 min at 1.5 bar and 121° C.) or by filter sterilization. The components may be sterilized either together or, if required, separately. All media components may be present at the start of the cultivation or added continuously or batchwise, as desired.

The culture temperature is normally between 15° C. and 45° C., preferably at from 25° C. to 40° C., and may be kept constant or may be altered during the experiment. The pH of the medium should be in the range from 5 to 8.5, preferably around 7.0. The pH for cultivation can be controlled during cultivation by adding basic compounds such as sodium hydroxide, potassium hydroxide, ammonia and aqueous ammonia or acidic compounds such as phosphoric acid or sulfuric acid. Foaming can be controlled by employing antifoams such as, for example, fatty acid polyglycol esters. To maintain the stability of plasmids it is possible to add to the medium suitable substances having a selective effect, for example antibiotics. Aerobic conditions are maintained by introducing oxygen or oxygen-containing gas mixtures such as, for example, ambient air into the culture. The fermentation broths obtained in this way, in particular those comprising polyunsaturated fatty acids, usually contain a dry mass of from 7.5 to 25% by weight.

The fermentation broth can then be processed further. The biomass may, according to requirement, be removed completely or partially from the fermentation broth by separation methods such as, for example, centrifugation, filtration, decanting or a combination of these methods or be left completely in said broth. It is advantageous to process the biomass after its separation.

However, the fermentation broth can also be thickened or concentrated without separating the cells, using known methods such as, for example, with the aid of a rotary evaporator, thin-film evaporator, falling-film evaporator, by reverse osmosis or by nanofiltration. Finally, this concentrated fermentation broth can be processed to obtain the fatty acids present therein

According to a further aspect of the invention there is provided a method to screen for glycosyltransferase enzymes that modify a substrate with rhamnose comprising the steps of:
i) providing a cell culture comprising a cell according to the invention wherein said cell is transformed or transfected with a nucleic acid molecule that encodes a glycosyltransferase enzyme to be tested, a substrate to be modified and rhamnose;
ii) culturing said cell under cell culture conditions that facilitate the production of a rhamnose modified substrate; and
iii) detecting the presence or not of said rhamnose modified substrate.

In a preferred method of the invention said glycosyltransferase is selected from the group consisting of: glucosyltransferase; fucosyltransferase; sialyltransferase; galactosyltransferases; glucuronosyltransferases; rhamnosyltransferases; and mannosyltransferases.

In a preferred method of the invention said glycosyltransferase is a glucosyltransferase; preferably a plant glucosyltransferase.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

An embodiment of the invention will now be described by example only and with reference to the following Figures:

FIG. 1a is the nucleotide and amino acid sequence of RHM1; FIG. 1b is the nucleotide and amino acid sequence of RHM2 and FIG. 1c is the nucleotide and amino acid sequence of RHM3;

FIG. 2a is the nucleotide and amino acid sequence of 78D1; FIG. 2b is the nucleotide and amino acid sequence of 89C1;

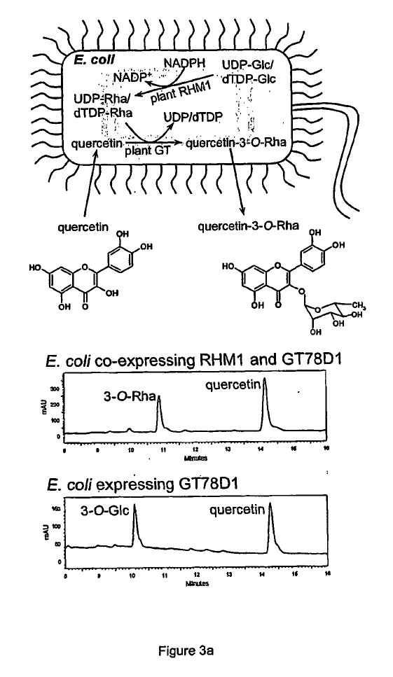

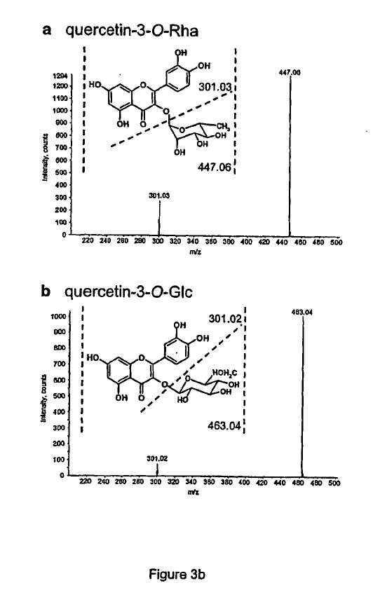

FIG. 3A Synthesis of quercetin-3-O-rhamnoside through whole-cell biocatalysis. (a) E. coli whole-cell biocatalysis system involving co-expression of Arabidopsis RHM and GT genes. Quercetin-3-O-rhamnoside (b) was formed in the whole-cell system co-expressing Arabidopsis RHM and GT78D1 genes whilst quercetin-3-O-glucoside (c) was produced when the whole-cell system only expressed Arabidopsis GT78D1. The MS/MS spectra of these glycosides are shown in FIG. 3B.

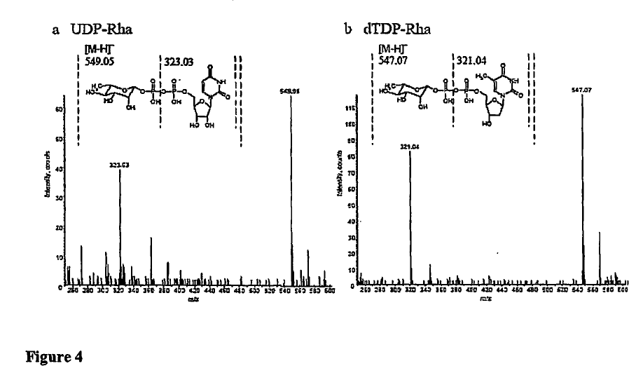

FIG. 4. MS analysis of (a) UDP-Rha and (b) dTDP-Rha produced by the whole-cell biocatalysis system expressing RHM1.

### METHODS

### Plasmid Construction

The cDNAs of RHM1, RHM2 and RHM3 were amplified from an Arabidopsis root cDNA library (courtesy of Dr Tobias Sieberer, University of York) by PCR using the primers listed in Table 1. The cDNAs were cloned into pGEX-2T vector (Amersham Pharmacia) using the BamHI and SmaI (RHM1) or EcoRI (RHM2 and RHM3) sites. The resulting plasmids allow the RHM proteins to be expressed as recombinant proteins with a glutathione-S-transferase (GST) fusion at the N-terminus. For combinatorial rhamnoside biosynthesis, the RHM cDNAs were cloned into the BamHI and XhoI (RHM1) or EcoRI (RHM2 and RHM3) sites of pET-28a vector (Novagen) which contains a kanamycin resistance gene.

### Recombinant Protein Purification and Activity Assay.

The plasmids expressing GST-RHM fusion proteins were transformed into E. coli BL21 cells for recombinant protein preparation. The cells were grown at 20° C. in 75 ml 2×YT medium containing 50 μg/ml ampicillin to an OD₆₀₀ of 0.8-1.0. The culture was then incubated with 1 mM isopropyl-1-thio-β-D-galactopyranoside (IPTG) for 24 h at 20° C. The cells were harvested (5000 g for 5 min), osmotically shocked centrifuged again (40,000 g for 30 min). The supernatant was mixed with 100 μl of 50% glutathione-coupled Sepharose (Pharmacia) at room temperature for 30 min. The beads were then washed with phosphate-buffered saline and adsorbed proteins were eluted with 20 mM reduced-form glutathione, 100 mM Tris-HCl (pH 8.0), and 120 mM NaCl, according to the manufacturer's instructions. The activity of RHM recombinant protein was assayed following the methods described by Barber with modification. Each reaction mix (200 μl) contained 10 mM Tris-HCl (pH 8.0), 14 mM 2-mercaptoethanol, 0.5 mM UDP- or dTDP-glucose, 1.25 mM NADPH and 10 μg recombinant proteins. The reaction was carried out at 37° C. for 1 h. The reaction mix was stored at −20° C. before HPLC-MS analysis.

### Combinatorial Whole-Cell Biocatalysis.

The plasmids pGEX-2T-GT and pET-28a-RHM were co-transformed into E. coli BL21 cells for whole-cell biosynthesis of quercetin rhamnoside. The transformed cells were selected on 2×YT plates containing 50 μg/ml ampicillin and 50 μg/ml kanamycin. Single colonies were picked into 10 ml 2×YT medium and were incubated at 37° C. overnight. The cells were then washed with fresh medium and were diluted to an OD₆₀₀ of 0.7. After the addition of 1 mM IPTG, the bacterial cultures were incubated at 28° C. for 6 h. To synthesise quercetin rhamnoside, 1 mM quercetin aglycone was added into the culture medium, and the cells were incubated at 28° C. for 24 h. The culture medium was then collected through centrifugation, and was extracted with butanol to purify the rhamnoside produced by the whole-cell biocatalysis.

### HPLC-MS Analysis.

An ion-pair HPLC-MS method was used to analyze nucleotide sugars. The ion-pair HPLC was carried out using an Agilent 1100 HPLC system. The samples were analyzed using a Columbus 5-μm C18 column (150×3.2 mm, Phenomenex) at a flow rate of 0.5 ml/min with isocratic 20 mM triethylammoniumacetate (TEAA) buffer (pH 6.0) for 15 min, followed by a linear gradient of 0-2% acetonitrile in 20 mM TEAA buffer over 20 min. The column was then washed with 4% acetonitrile in 20 mM TEAA buffer for 5 min and equilibrated with 20 mM TEAA buffer for 5 min. The chromatography was monitored at 260 nm. Negative ion electrospray MS and MS/MS data were acquired on an Applied Biosystems QSTAR Pulsar i hybrid quadropole time-of-flight instrument, scanning the ranges m/z 250-650. Nitrogen was used as nebulisation gas (3.3 L/min). The capillary temperature was set at 300° C. with an ion spray voltage of −2500 V. For MS/MS study, either −10 or −30 V of collision energy was applied. The data were collected and processed using ANALYST QS (Applied Biosystems) software.

The quercetin glycosides formed in the whole-cell biocatalysis were analyzed using a reverse-phase HPLC-MS method. The reverse-phase HPLC was carried out using the system described above with a different mobile phase. A linear gradient of 10-50% acetonitrile in 0.1% trifluoroacetic acid (TFA) buffer over 15 min followed by an increase to 80% acetonitrile in 0.1% TFA buffer in 10 min was used. The column was then washed with 100% acetonitrile buffer for 5 min and equilibrated with 10% acetonitrile buffer for 5 min. The MS/MS study was carried as described above with the collision energy set at −20, −40, and −60 V.

TABLE 1
Summary of primers designed for plasmid construction

| | | |
|---|---|---|
| Primer name | Restriction site | Sequence (5′-3′) |

| | | |
|---|---|---|
| RHM1 forward | BamHI | CGGGATCCATGGCTTCGTACACTCCC |
| RHM1 reverse | XhoI | CCGCTCGAGTCAGGTTTTCTTGTTTGGC |
| RHM2 forward | BamHI | CGGGATCCATGGATGATACTACGTATAA |
| RHM2 reverse | EcoRI | CGGAATTCTTAGGTTCTCTTGTTTGG |
| RHM3 forward | BamHI | CGGGATCCATGGCTACATATAAGCCTAA |
| RHM3 reverse | EcoRI | CGGAATTCTTACGTTCTCTTGTTAGGTT |

Restriction sites within the primers are underlined for clarity.

Preparation of UDP-Rha and dTDP-Rha from E. coli cells. E. coli cells expressing RHM proteins were harvested and disrupted by French Press (ThermoElectron) in 10 ml PBS buffer. After centrifugation (40,000×g for 5 min), the supernatant was collected, filtrated through Biomax-30K and Biomax-10K (Millipore), and freeze-dried. The sample was dissolved in H₂O and was analyzed by ion-pair HPLC-MS.

### EXAMPLES

UDP-Rha is a ubiquitous nucleotide sugar in plants. Whilst the enzyme(s) involved in UDP-Rha biosynthesis in plants has not been characterised in detail, in microorganisms three enzymes are known to convert dTDP-Glc to dTDP-Rha. These include dTDP-Glc 4,6-dehydratase (rm1B), dTDP-4-keto-6-deoxy-Glc 3,5-epimerase (rm1C) and dTDP-4-keto-Rha reductase (rm1D). In Arabidopsis thaliana three sequences were found to encode proteins RHM1, RHM2 and RHM3 each with an N-terminal domain containing amino acid sequence similar to the bacterial 4,6-dehydratase and a C-terminal domain partly similar to the bacterial 3,5-epimerase and partly similar to the bacterial 4-keto-reductase. Since these RHM proteins are potentially capable of catalysing three different reactions and converting UDP-Glc to UDP-Rha per se, expression of these proteins in E. coli may result in the accumulation of UDP-Rha, which is then available for GTs to rhamnosylate of small molecules.

To examine the catalytic activities of these RHM proteins, all three corresponding cDNA were amplified from an arabidopsis root cDNA library, and were subcloned into pGEX-2T vector for recombinant protein preparation. When incubated in vitro with dTDP-Glc or UDP-Glc and the co-factor NADPH, the recombinant RHMs were found to be able to form the corresponding nucleotide-Rha (FIG. 4). Although the biosynthesis of dTDP/UDP-Rha from dTDP/UDP-Glc is likely to involve three reaction steps, no intermediates were observed in our study. This is in contrast to the reactions carried out using plant protein extracts in which an intermediate 4-keto-6-deoxy-Glc was reported.

In the cell lysate of untransformed E. coli BL21, only trace amount of UDP-Glc, dTDP-Glc and dTDP-Rha were detected (<μg/L culture). In contrast, when the cells expressed the RHM cDNAs, the lysate contained a significant level of UDP- and dTDP-Rha with no changes in the levels of UDP- and dTDP-Glc. These results confirmed the enzymes are able to use both UDP- and dTDP-Glc as substrates.

Several combinatorial whole-cell systems have been reported for the biosynthesis of oligosaccharides and polymethylated quercetin. These systems involve two or more bacterial strains expressing different proteins. In order to develop a simple whole-cell rhamnosylation system, in this study, the bacterial cells were co-transformed with the RHM1 cDNA and a GT, for example, UGT78D1.

After 24 h of incubation with quercetin, the bacterial cells co-expressing RHM1 and UGT78D1 were found to form quercetin rhamnoside, whereas in the bacterial culture expressing UGT78D1, only 3-O-glucoside of quercetin was obtained (FIG. 3a). The production of the rhamnoside in the combinatorial whole-cell system may be due to a higher level of UDP-Rha present in the cells, or a higher affinity of the GT towards UDP-Rha than UDP-Glc. Nevertheless, the bacterial cells expressing plant RHM1 and GT proteins have proved to be an efficient system for rhamnosylation of small molecules. It is as yet unclear whether UGT78D1 used only UDP-Rha as the donor, or transferred Rha from both UDP-Rha and dTDP-Rha to the acceptor molecule in the cells. When we analyzed the activity of UGT78D2, which is highly homologous to UGT78D1, the GT was capable of using both UDP-Glc and dTDP-Glc as donors. Furthermore, in the two GT protein structures from the same family that were recently solved, the nucleotide-binding pocket, which is conserved in this family of GTs, does not have any features to discriminate between UDP-sugar and dTDP-sugar. Thus, it is likely that UGT78D1 used both UDP- and dTDP-Rha as donor for rhamnosylation in the combinatorial whole-cell system.

The whole-cell system developed in this study not only can be used to synthesize UDP-Rha, dTDP-Rha, and rhamnosides, it also provides a platform to explore the activity of other rhamnosyltransferases from a GT enzyme library. In our previous study, we reported a total number of 107 GTs for small molecules present in arabidopsis. Over ninety of these GTs had been analyzed against two substrates, quercetin and esculetin, using UDP-Glc as the donor. The GTs glycosylating these two compounds numbered 29 and 48 respectively. The capability of the GTs in this family in utilizing UDP-Rha was not investigated due to the lack of the donor. The whole-cell system developed in this study made this screening experiment possible. The entire family of GTs was co-expressed individually with RHM1, and was screened in the whole-cell system for activity towards quercetin and esculetin. This led to the identification of a GT, UGT89C1, capable of using UDP/dTDP-Rha as donors to form quercetin rhamnoside.

Several methods have been developed to synthesize nucleotide sugars such as UDP-Glc and UDP-Gal using isolated enzymes. These systems often involve multiple enzymes to regenerate the co-factors and therefore the cost for production can be high. Chemical synthesis of these nucleotide sugars is also sophisticated and involves multiple reaction steps. In contrast to these approaches, in this study, we reported a simple whole-cell system for synthesis of UDP- and dTDP-Rha without supplementary co-factor NADPH. Use of the plant enzymes allow all the reaction steps to be catalysed by one enzyme species.

### REFERENCES

1. Burger, A., Berendes, R., Voges, D., Huber, R. & Demange, P. A rapid and efficient purification method for recombinant annexin V for biophysical studies. FEBS Lett. 329, 25-28 (1993).
2. Li, Y., Baldauf, S., Lim, E.-K. & Bowles, D. J. Phylogenetic analysis of the UDP-glycosyltransferase multigene family of Arabidopsis thaliana. J. Biol. Chem. 276, 4338-4343 (2001).
3. Kamsteeg, J., Van Brederode, J. & Van Nigtevecht, G. The formation of UDP-L-rhamnose from UDP-D-glucose by an enzyme preparation of red campion (Szlene dzoica (L) clairv) leaves. FEBS Lett. 91, 281-284 (1978).
4. Räbinä, J., Mäki, M., Savilahti, E. M., Järvinen, N., Penttilä, L. & Renkonen, R. Analysis of nucleotide sugars from cell lysates by ion-pair solid-phase extraction and reverse-phase high-performance liquid chromatography. Glycoconjugate J. 18, 799-805 (2001).

1. A prokaryotic cell that is transfected with at least one nucleic acid molecule that comprises a nucleic acid sequence selected from the group consisting of:
i) SEQ ID NO: 1, 3 and 5; and
ii) sequences that hybridize under stringent hybridization conditions to a sequence of SEQ ID NO: 1, 3 or 5 and which have rhamnose synthase activity.

2. A cell according to claim 1 wherein said cell is further transfected with a nucleic acid molecule comprising a nucleic acid sequence of SEQ ID NO: 7, 9 or 10; or a nucleic acid molecule comprising a nucleic acid sequence that hybridizes under stringent hybridization conditions to a sequence of SEQ ID NO: 7, 9 or 10 and which has glucosyltransferase activity.

3. A cell according to claim 1 wherein said prokaryotic cell is a bacterial cell.

4. A cell according to claim 1 wherein said nucleic acid molecule comprises the nucleic acid sequence of SEQ ID NO: 1, 3 or 5.

5. A cell according to claim 1 wherein said nucleic acid molecule consists of the nucleic acid sequence of SEQ ID NO: 1, 3 or 5.

6. A cell according to claim 2 wherein said nucleic acid molecule comprises the nucleic acid sequence of SEQ ID NO: 7, 9 or 10.

7. A cell according to claim 2 wherein said nucleic acid molecule consists of the nucleic acid sequence of SEQ ID NO: 7, 9 or 10.

8. A cell according to claim 1 wherein said cell is transfected with an expression vector that includes said nucleic acid molecules that encode said rhamnose synthase and said glucosyltransferase polypeptides.

9. A cell culture comprising a cell according to claim 1.

10-11. (canceled)

12. A method for the production of a nucleotide sugar comprising the steps of:
i) providing a cell culture according to claim 9 and rhamnose; and
ii) culturing said cell under cell culture conditions that facilitate the production of a nucleotide sugar wherein said nucleotide sugar is UDP-rhamnose.

13. A method for the production of a substrate which is modified with a rhamnoside sugar comprising the steps of
i) providing a cell culture according to claim 9 and at least one substrate to be modified; and
ii) culturing said cell under cell culture conditions that facilitate the production of a sugar modified substrate.

14. A method according to claim 13 wherein said substrate is quercetin.

15. A reaction vessel comprising a cell according to claim 1.

16. A reaction vessel according to claim 15, wherein the vessel is a bioreactor.

17. A reaction vessel according to claim 15 wherein said bioreactor comprises nutrient media that does not include an exogenous supply of UDP-glucose.

18. A method to screen for glycosyltransferase enzymes that modify a substrate with rhamnose comprising the steps of:
i) providing a cell culture comprising a cell according to claim 1 wherein said cell is transformed or transfected with a nucleic acid molecule that encodes a glycosyltransferase enzyme to be tested, a substrate to be modified and rhamnose;
ii) culturing said cell under cell culture conditions that facilitate the production of a rhamnose modified substrate; and
iii) detecting the presence or not of said rhamnose modified substrate.

19. A method according to claim 18 wherein said glycosyltransferase is selected from the group consisting of: glucosyltransferase; fucosyltransferase; sialyltransferase; galactosyltransferases; glucuronosyltransferases; rhamnosyltransferases; and mannosyltransferases.

20. A method according to claim 19 wherein said glycosyltransferase is a glucosyltransferase.

21. A method according to claim 19 wherein said glycosyltransferase is a plant glycosyltransferase.

22. The method of claim 12 further comprising separating or purifying UDP rhamnose from the cell or cell culture media.

23. The method of claim 13 further comprising separating or purifying said sugar modified substrate from the cell or cell culture media.


SEQUENCE LISTING (17 sequences)

SEQ ID NO: 1 - DNA, 2010 bp, Arabidopsis thaliana
atggcttcgtacactcccaagaacattctcatcaccggagctgctggtttcattgcgtctcatgtcgccaacagactcatacgaagctatcctgattacaaaatcgttgtgcttgacaagcttgattactgttcaaatctcaagaatctcaatccttctaagcactctccgaacttcaagtttgtcaaaggtgatatcgctagtgctgacttggtgaatcatcttctcatcactgaaggtattgacaccatcatgcatttcgctgctcagactcacgtcgacaattccttcggtaacagtttcgagtttactaagaataatatctatggaactcatgtccttcttgaggcttgtaaagttactggtcagattaggaggtttattcatgttagtactgatgaagtttatggtgaaactgatgaggatgctcttgttggtaaccatgaggcttctcagctgcttccgacgaatccttactctgccacgaaagctggtgctgagatgcttgttatggcttatggtagatcttatggtttgcctgttattaccactcgtgggaataacgtctatggaccgaatcagtttcctgagaagttgattcctaagttcattttgctggcaatgagagggcaggttcttcccattcatggagatggatcaaatgtcaggagctacctctactgtgaagacgttgctgaggcttttgaagttgttcttcacaagggagaagttggccatgtttacaatattgggacgaagaaggagaggagagtgaatgatgttgccaaagacatctgcaaactcttcaacatggaccctgaggcgaacatcaagtttgtcgacaacagaccttttaacgatcagaggtacttccttgacgatcagaagctcaaaaagttgggatggtcagagagaaccacgtgggaagaagggttgaagaaaactatggattggtacacacagaacccggagtggtggggtgatgtttctggagcattgcttcctcatccaaggatgctgatgatgcctggtgggcgacactttgatggctccgaggacaattcgctggcagctactttatctgaaaaaccaagtcaaacccatatggttgttccaagccaaaggagcaacggcacacctcaaaagccttcgctgaagttcctgatatatggaaagaccggatggatcggtggtctgcttggaaagatatgtgataagcaaggaattgcttacgagtatgggaaaggtcggttggaggatcgatcttctcttctgcaggatattcagagtgttaagccaacccatgttttcaattccgctggtgtgactgggagacccaatgttgactggtgtgagtctcacaagaccgagactatccgtgccaatgtagctggcacattgactctagctgatgtctgcagagagcacggactcctaatgatgaatttcgctactggttgtatattcgaatatgacgacaagcatccggaaggttcaggaattggcttcaaggaggaagacacacccaacttcactggctctttctactcgaaaaccaaagccatggtcgaggagctgctaaaggagtatgacaacgtatgcacattgagggtaaggatgccgatctcctcggatctaaacaacccgcgcaacttcatcaccaagatctccaggtacaacaaagtagtgaacatcccaaacagcatgactgtgttggacgagttattaccaatctccatcgagatggcgaaaagaaacttgaaaggaatctggaacttcacaaacccaggtgtggtgagccacaacgagatcctagagatgtacagagactacatcaaccctgaattcaaatgggcaaacttcacattagaggagcaagctaaagtcattgtggctccaagaagcaacaacgagatggatgcttccaagctcaagaaagagttccctgagctactctctatcaaggagtctctgattaagtatgcatacgggccaaacaagaaaacctga

SEQ ID NO: 2 - protein, 669 aa, Arabidopsis thaliana
MASYTPKNILITGAAGFIASHVANRLIRSYPDYKIVVLDKLDYCSNLKNLNPSKHSPNFKFVKGDIASADLVNHLLITEGIDTIMHFAAQTHVDNSFGNSFEFTKNNIYGTHVLLEACKVTGQIRRFIHVSTDEVYGETDEDALVGNHEASQLLPTNPYSATKAGAEMLVMAYGRSYGLPVITTRGNNVYGPNQFPEKLIPKFILLAMRGQVLPIHGDGSNVRSYLYCEDVAEAFEVVLHKGEVGHVYNIGTKKERRVNDVAKDICKLFNMDPEANIKFVDNRPFNDQRYFLDDQKLKKLGWSERTTWEEGLKKTMDWYTQNPEWWGDVSGALLPHPRMLMMPGGRHFDGSEDNSLAATLSEKPSQTHMVVPSQRSNGTPQKPSLKFLIYGKTGWIGGLLGKICDKQGIAYEYGKGRLEDRSSLLQDIQSVKPTHVFNSAGVTGRPNVDWCESHKTETIRANVAGTLTLADVCREHGLLMMNFATGCIFEYDDKHPEGSGIGFKEEDTPNFTGSFYSKTKAMVEELLKEYDNVCTLRVRMPISSDLNNPRNFITKISRYNKVVNIPNSMTVLDELLPISIEMAKRNLKGIWNFTNPGVVSHNEILEMYRDYINPEFKWANFTLEEQAKVIVAPRSNNEMDASKLKKEFPELLSIKESLIKYAYGPNKKT

SEQ ID NO: 3 - DNA, 2004 bp, Arabidopsis thaliana
atggatgatactacgtataagccaaagaacattctcattactggagctgctggatttattgcttctcatgttgccaacagattaatccgtaactatcctgattacaagatcgttgttcttgacaagcttgattactgttcagatctgaagaatcttgatccttctttttcttcaccaaatttcaagtttgtcaaaggagatatcgcgagtgatgatctcgttaactaccttctcatcactgaaaacattgatacgataatgcattttgctgctcaaactcatgttgataactcttttggtaatagctttgagtttaccaagaacaatatttatggtactcatgttcttttggaagcctgtaaagttacaggacagatcaggaggtttatccatgtgagtaccgatgaagtctatggagaaaccgatgaggatgctgctgtaggaaaccatgaagcttctcagctgttaccgacgaatccttactctgcaactaaggctggtgctgagatgcttgtgatggcttatggtagatcatatggattgcctgttattacgactcgcgggaacaatgtttatgggcctaaccagtttcctgaaaaaatgattcctaagttcatcttgttggctatgagtgggaagccgcttcccatccatggagatggatctaatgtccggagttacttgtactgcgaagacgttgctgaggcttttgaggttgttcttcacaaaggagaaatcggtcatgtctacaatgtcggcacaaaaagagaaaggagagtgatcgatgtggctagagacatctgcaaacttttcgggaaagaccctgagtcaagcattcagtttgtggagaaccggccctttaatgatcaaaggtacttccttgatgatcagaagctgaagaaattggggtggcaagagcgaacaaattgggaagatggattgaagaagacaatggactggtacactcagaatcctgagtggtggggtgatgtttctggagctttgcttcctcatccgagaatgcttatgatgcccggtggaagactttctgatggatctagtgagaagaaagacgtttcaagcaacacggtccagacatttacggttgtaacacctaagaatggtgattctggtgacaaagcttcgttgaagtttttgatctatggtaagactggttggcttggtggtcttctagggaaactatgtgagaagcaagggattacatatgagtatgggaaaggacgtctggaggatagagcttctcttgtggcggatattcgtagcatcaaacctactcatgtgtttaatgctgctggtttaactggcagacccaacgttgactggtgtgaatctcacaaaccagagaccattcgtgtaaatgtcgcaggtactttgactctagctgatgtttgcagagagaatgatctcttgatgatgaacttcgccaccggttgcatctttgagtatgacgctacacatcctgagggttcgggtataggtttcaaggaagaagacaagccaaatttctttggttctttctactcgaaaaccaaagccatggttgaggagctcttgagagaatttgacaatgtatgtaccttgagagtccggatgccaatctcctcagacctaaacaacccgagaaacttcatcacgaagatctcgcgctacaacaaagtggtggacatcccgaacagcatgaccgtactagacgagcttctcccaatctctatcgagatggcgaagagaaacctaagaggcatatggaatttcaccaacccaggggtggtgagccacaacgagatattggagatgtacaagaattacatcgagccaggttttaaatggtccaacttcacagtggaagaacaagcaaaggtcattgttgctgctcgaagcaacaacgaaatggatggatctaaactaagcaaggagttcccagagatgctctccatcaaagagtcactgctcaaatacgtctttgaaccaaacaagagaacctaa

SEQ ID NO: 4 - protein, 667 aa, Arabidopsis thaliana
MDDTTYKPKNILITGAAGFIASHVANRLIRNYPDYKIVVLDKLDYCSDLKNLDPSFSSPNFKFVKGDIASDDLVNYLLITENIDTIMHFAAQTHVDNSFGNSFEFTKNNIYGTHVLLEACKVTGQIRRFIHVSTDEVYGETDEDAAVGNHEASQLLPTNPYSATKAGAEMLVMAYGRSYGLPVITTRGNNVYGPNQFPEKMIPKFILLAMSGKPLPIHGDGSNVRSYLYCEDVAEAFEVVLHKGEIGHVYNVGTKRERRVIDVARDICKLFGKDPESSIQFVENRPFNDQRYFLDDQKLKKLGWQERTNWEDGLKKTMDWYTQNPEWWGDVSGALLPHPRMLMMPGGRLSDGSSEKKDVSSNTVQTFTVVTPKNGDSGDKASLKFLIYGKTGWLGGLLGKLCEKQGITYEYGKGRLEDRASLVADIRSIKPTHVFNAAGLTGRPNVDWCESHKPETIRVNVAGTLTLADVCRENDLLMMNFATGCIFEYDATHPEGSGIGFKEEDKPNFFGSFYSKTKAMVEELLREFDNVCTLRVRMPISSDLNNPRNFITKISRYNKVVDIPNSMTVLDELLPISIEMAKRNLRGIWNFTNPGVVSHNEILEMYKNYIEPGFKWSNFTVEEQAKVIVAARSNNEMDGSKLSKEFPEMLSIKESLLKYVFEPNKRT

SEQ ID NO: 5 - DNA, 1995 bp, Arabidopsis thaliana
atggctacatataagcctaagaacatcctcatcactggggctgctggattcatagcctctcatgttgctaacagactagttcgcagctaccctgactacaaaattgttgtgcttgacaagcttgattactgttctaatctgaagaaccttaatccttctaaatcctctcccaacttcaagtttgtgaaaggagatatcgccagtgctgatctcgtcaactaccttctcatcactgaagaaatcgacaccattatgcactttgctgctcaaacccatgttgacaattctttcggtaatagctttgagtttaccaagaacaatatttatggtacccatgtccttttggaagcttgtaaagtcactggccagatcaggaggttcatccatgtgagtactgatgaggtctatggagagactgatgaggatgcttcagtgggtaatcacgaggcttctcagttgctcccaactaatccatactccgccactaaagctggagctgagatgcttgtcatggcatatggtagatcatatgggttgccggttataacaactcgcgggaacaatgtttatggtcctaaccagtttcctgaaaagttgattcctaagttcatcctcttggccatgaatgggaagcctctcccaatccacggagatggatctaatgtgagaagttatctctactgcgaagatgttgctgaggcatttgaggttgttcttcacaaaggggaagttaaccatgtctacaatatagggacaacgagagaaaggagagtgattgatgtggctaatgacatcagtaaactctttgggatagaccctgactccaccattcagtatgtggaaaaccggccattcaatgaccagaggtacttcctcgatgaccagaagctgaagaaattaggatggtgtgagcgaaccaattgggaagaaggactgaggaagacaatggaatggtatactgagaaccctgagtggtggggcgatgtttctggagctctgcttcctcatccacggatgttgatgatgcccggtgaccgacactctgatggctctgacgagcacaagaatgcagatggtaatcagacattcacggtggttactcccaccaaggctggttgttccggagacaaaagatccttgaagttcctcatctatgggaagactgggtggctcggtggtcttctgggaaaactatgtgagaaacaagggattccttacgagtatggaaaaggaagactagaggatagagcttctctcatcgcagatattcgcagcatcaaaccaagtcatgtcttcaacgccgctggtttaactgggagacccaatgttgactggtgtgaatctcacaaaactgaaaccatccgagtcaacgttgctggaactttgactcttgcagatgtttgcagagagaatgatctgttgatgatgaactttgccactggttgtatattcgagtatgacgctgcacatccagaaggttcagggattggttttaaggaagaagataaaccgaatttcactggttctttctactcaaaaacaaaggcaatggtggaagagcttctaagagaatttgacaacgtatgcaccttgagagtgcggatgccaatctcatctgacttaaataacccgcgaaacttcatcacgaagatctcgcgttacaacaaagtggtgaacattccaaacagcatgaccatactagatgaactcttaccaatctcgatcgagatggcgaagaggaacctaaggggaatatggaacttcaccaatccaggagtggtgagccacaacgagatattagagatgtacaagagttacatcgagcctgatttcaaatggtccaacttcaatttggaagaacaggctaaggtcattgttgctccacggagcaacaacgagatggatggtgccaagctcagcaaggagtttccagagatgctttccatcaaagattcgttgatcaaatacgtcttcgaacctaacaagagaacgtaa

SEQ ID NO: 6 - protein, 664 aa, Arabidopsis thaliana
MATYKPKNILITGAAGFIASHVANRLVRSYPDYKIVVLDKLDYCSNLKNLNPSKSSPNFKFVKGDIASADLVNYLLITEEIDTIMHFAAQTHVDNSFGNSFEFTKNNIYGTHVLLEACKVTGQIRRFIHVSTDEVYGETDEDASVGNHEASQLLPTNPYSATKAGAEMLVMAYGRSYGLPVITTRGNNVYGPNQFPEKLIPKFILLAMNGKPLPIHGDGSNVRSYLYCEDVAEAFEVVLHKGEVNHVYNIGTTRERRVIDVANDISKLFGIDPDSTIQYVENRPFNDQRYFLDDQKLKKLGWCERTNWEEGLRKTMEWYTENPEWWGDVSGALLPHPRMLMMPGDRHSDGSDEHKNADGNQTFTVVTPTKAGCSGDKRSLKFLIYGKTGWLGGLLGKLCEKQGIPYEYGKGRLEDRASLIADIRSIKPSHVFNAAGLTGRPNVDWCESHKTETIRVNVAGTLTLADVCRENDLLMMNFATGCIFEYDAAHPEGSGIGFKEEDKPNFTGSFYSKTKAMVEELLREFDNVCTLRVRMPISSDLNNPRNFITKISRYNKVVNIPNSMTILDELLPISIEMAKRNLRGIWNFTNPGVVSHNEILEMYKSYIEPDFKWSNFNLEEQAKVIVAPRSNNEMDGAKLSKEFPEMLSIKDSLIKYVFEPNKRT

SEQ ID NO: 7 - DNA, 1362 bp, Arabidopsis thaliana
atgaccaaattctccgagccaatcagagactcccacgtggcagttctcgcgtttttccccgttggcgctcatgccggtcctctcttagccgtcactcgccgtctcgccgccgcttctccctccaccatcttttctttcttcaacaccgcaagatcaaacgcgtcgttgttctcctctgatcatcccgagaacatcaaggtccacgacgtctctgacggtgttccggagggaaccatgctcgggaatccactggagatggtcgagctgtttctcgaagcggctccacgtattttccggagcgaaatcgcggcggcagagatagaagttggaaagaaagtgacatgcatgctaacagatgccttcttctggttcgcagcggacatagcggctgagctgaacgcgacttgggttgccttctgggccggcggagcaaactcactctgtgctcatctctacactgatctcatcagagaaaccatcggtctcaaagatgtgagtatggaagagacattagggtttataccaggaatggagaattacagagttaaagatataccagaggaagttgtatttgaagatttggactctgttttcccaaaggctttataccaaatgagtcttgctttacctcgtgcctctgctgttttcatcagttcctttgaagagttagaacctacattgaactataacctaagatccaaacttaaacgtttcttgaacatcgcccctctcacgttattatcttctacatcggagaaagagatgcgtgatcctcatggctgctttgcttggatggggaagagatcagctgcttctgtagcgtacattagcttcggcaccgtcatggaacctcctcctgaagagcttgtggcgatagcacaagggttggaatcaagcaaagtgccgtttgtttggtcgctgaaggagaagaacatggttcatctaccaaaagggtttttggatcggacaagagagcaagggatagtggttccttgggctccacaagtggaactgctgaaacacgaggcaatgggtgtgaatgtgacacattgtggatggaactcagtgttggagagtgtgtcggcaggtgtaccgatgatcggcagaccgattttggcggataataggctcaacggaagagcagtggaggttgtgtggaaggttggagtgatgatggataatggagtcttcacgaaagaaggatttgagaagtgtttgaatgatgtttttgttcatgatgatggtaagacgatgaaggctaatgccaagaagcttaaagaaaaactccaagaagatttctccatgaaaggaagctctttagagaatttcaaaatattgttggacgaaattgtgaaagtttag

SEQ ID NO: 8 - protein, 453 aa, Arabidopsis thaliana
MTKFSEPIRDSHVAVLAFFPVGAHAGPLLAVTRRLAAASPSTIFSFFNTARSNASLFSSDHPENIKVHDVSDGVPEGTMLGNPLEMVELFLEAAPRIFRSEIAAAEIEVGKKVTCMLTDAFFWFAADIAAELNATWVAFWAGGANSLCAHLYTDLIRETIGLKDVSMEETLGFIPGMENYRVKDIPEEVVFEDLDSVFPKALYQMSLALPRASAVFISSFEELEPTLNYNLRSKLKRFLNIAPLTLLSSTSEKEMRDPHGCFAWMGKRSAASVAYISFGTVMEPPPEELVAIAQGLESSKVPFVWSLKEKNMVHLPKGFLDRTREQGIVVPWAPQVELLKHEAMGVNVTHCGWNSVLESVSAGVPMIGRPILADNRLNGRAVEVVWKVGVMMDNGVFTKEGFEKCLNDVFVHDDGKTMKANAKKLKEKLQEDFSMKGSSLENFKILLDEIVKV

SEQ ID NO: 9 - DNA, 1308 bp, Arabidopsis thaliana
ttacaaacacatctctgcaacgagctcatccaagttcttgtaagagctcccaccttctttaatggcctccatagctttctccctcagcttcatcaacgtaactctctccggcaagtcctctctcgccgactcagccaaaatcctagcgagcttgtccgagtcaggaaccgagtctctgttctctccaactcgcactgcggctcttagtttatcaacgatgagcgtcgtgttaaagaaatggtctgcttgcatcggccacgctagcaacataactcctccgaccattccttccagaaccgaaccccaacccaaatgagttaggtaagatccaacggctcgatgctcaagaatcatagtttgtggggcccatcctcttatcacgagtcctttctccttcactctctcttcaaatcccgccgggatcacatcttcctcaacggagttatcgctggagttcaccttcttagctgcgtctctcaccgcccatatgaaacgcacactgcttttctccaacgccgccgctaaagcagctgtttgctccgccgtgagccggatctggcttccaaaaccgacgtatacgacggagttatcctcggggcacgaatctaaccaagccgagactttcgccggcgggattgagctttgtccgccacggtcaacgccagctttaaaggggagcaacggtccgacggtccatatacggtggtgattcaggaaacgtgttttaacagtttctacaaactcaggctcgaggtcgtagaaactgttgatgacgagcccgtagctttccgttgtggcagtctcgagatcgttgaagaagcttctatcttcttgagcccacatgacggagatcgaatgagcattgatgggtaagaaactaatggacttaatagagaaagcatcagctactttgttaatccaagggctgagaaatgagcttcctaggatggcgtcggggagatccgacggtggttgacggctgagaaagtcaacgagagggtcgtggagacgagagagagcatcaaacatgtgaactatagcttcgagaggaagttgctggagagattcgacaccggaaggtatacaagggtgagaaggaaaaggaaggattagggttttgaagtgttccggggagtgaagagaacggagagcatcgagataggaagagtttttgggtgtgacgaggacagtgacggtggctccacggagaagaatctgatgcgtgaggtcaagatgtggaaccatgtgaccggattgtggaaacggtatcaccagaacgtgcggcttcttcgttgttgttgttgtcat

SEQ ID NO: 10 - DNA, 1308 bp, Arabidopsis thaliana
atgacaacaacaacaacgaagaagccgcacgttctggtgataccgtttccacaatccggtcacatggttccacatcttgacctcacgcatcagattcttctccgtggagccaccgtcactgtcctcgtcacacccaaaaactcttcctatctcgatgctctccgttctcttcactccccggaacacttcaaaaccctaatccttccttttccttctcacccttgtataccttccggtgtcgaatctctccagcaacttcctctcgaagctatagttcacatgtttgatgctctctctcgtctccacgaccctctcgttgactttctcagccgtcaaccaccgtcggatctccccgacgccatcctaggaagctcatttctcagcccttggattaacaaagtagctgatgctttctctattaagtccattagtttcttacccatcaatgctcattcgatctccgtcatgtgggctcaagaagatagaagcttcttcaacgatctcgagactgccacaacggaaagctacgggctcgtcatcaacagtttctacgacctcgagcctgagtttgtagaaactgttaaaacacgtttcctgaatcaccaccgtatatggaccgtcggaccgttgctcccctttaaagctggcgttgaccgtggcggacaaagctcaatcccgccggcgaaagtctcggcttggttagattcgtgccccgaggataactccgtcgtatacgtcggttttggaagccagatccggctcacggcggagcaaacagctgctttagcggcggcgttggagaaaagcagtgtgcgtttcatatgggcggtgagagacgcagctaagaaggtgaactccagcgataactccgttgaggaagatgtgatcccggcgggatttgaagagagagtgaaggagaaaggactcgtgataagaggatgggccccacaaactatgattcttgagcatcgagccgttggatcttacctaactcatttgggttggggttcggttctggaaggaatggtcggaggagttatgttgctagcgtggccgatgcaagcagaccatttctttaacacgacgctcatcgttgataaactaagagccgcagtgcgagttggagagaacagagactcggttcctgactcggacaagctcgctaggattttggctgagtcggcgagagaggacttgccggagagagttacgttgatgaagctgagggagaaagctatggaggccattaaagaaggtgggagctcttacaagaacttggatgagctcgttgcagagatgtgtttgtaa

SEQ ID NO: 11 - protein, 435 aa, Arabidopsis thaliana
MTTTTTKKPHVLVIPFPQSGHMVPHLDLTHQILLRGATVTVLVTPKNSSYLDALRSLHSPEHFKTLILPFPSHPCIPSGVESLQQLPLEAIVHMFDALSRLHDPLVDFLSRQPPSDLPDAILGSSFLSPWINKVADAFSIKSISFLPINAHSISVMWAQEDRSFFNDLETATTESYGLVINSFYDLEPEFVETVKTRFLNHHRIWTVGPLLPFKAGVDRGGQSSIPPAKVSAWLDSCPEDNSVVYVGFGSQIRLTAEQTAALAAALEKSSVRFIWAVRDAAKKVNSSDNSVEEDVIPAGFEERVKEKGLVIRGWAPQTMILEHRAVGSYLTHLGWGSVLEGMVGGVMLLAWPMQADHFFNTTLIVDKLRAAVRVGENRDSVPDSDKLARILAESAREDLPERVTLMKLREKAMEAIKEGGSSYKNLDELVAEMCL

SEQ ID NO: 12 - DNA, 26 bp, Arabidopsis thaliana
cgggatccatggcttcgtacactccc

SEQ ID NO: 13 - DNA, 28 bp, Arabidopsis thaliana
ccgctcgagtcaggttttcttgtttggc

SEQ ID NO: 14 - DNA, 28 bp, Arabidopsis thaliana
cgggatccatggatgatactacgtataa

SEQ ID NO: 15 - DNA, 26 bp, Arabidopsis thaliana
cggaattcttaggttctcttgtttgg

SEQ ID NO: 16 - DNA, 28 bp, Arabidopsis thaliana
cgggatccatggctacatataagcctaa

SEQ ID NO: 17 - DNA, 28 bp, Arabidopsis thaliana
cggaattcttacgttctcttgttaggtt